(19) United States
(12) Patent Application Publication
(10) Pub. No.: US 2009/0221655 A1
(43) Pub. Date: Sep. 3, 2009

(54) ANTIBACTERIAL AGENTS

(75) Inventors: Vara Prasad Venkata Nagendra Josyula, Superior Township, MI (US); Allison Laura Choy, Lexington, MA (US)

Correspondence Address: PFIZER INC;Mary J Hosley, 150 EAST 42ND STREET, MS: 150/02/E112, NEW YORK, NY, 10017-5612, US

(73) Assignee: Pitzer, Inc., Groton, CT (US)

(21) Appl. No.: 12/278,006

(22) PCT Filed: Jan. 22, 2007
(86) PCT No.: PCT/IB2007/000179

§ 371 Date: Jan. 7, 2009

Related U.S. Application Data
(60) Provisional application No. 60/764,113, filed on Feb. 1, 2006.

(51) Int. Cl. A61K 31/422; C07D 413/10; A61P 31/04

(52) U.S. Cl. 514/376; 548/231

(57) ABSTRACT

The present invention provides a compound of Formula (I) Or a pharmaceutically acceptable salt thereof wherein: W is CH₂NHC(═Z)R¹, C(═Z)NHR², or CH₂het; X is H, C₁₋₆alkyl, or C₂₋₆alkenyl; Y is H, or F; Z is O, or S; R¹ is C₁₋₆alkyl, NHC₁₋₆alkyl, C₃₋₇cycloalkyl, C₂₋₆alkenyl, or OC₁₋₄alkyl; R² is H, C₁₋₄alkyl, or —OC₁₋₄alkyl; and het is a five-(5) or six-(6) membered heterocyclic ring having 1-4 heteroatoms selected from the group consisting of oxygen, sulfur, and nitrogen within the ring, wherein each carbon atom in het is optionally substituted with C₁₋₄alkyl, C₂₋₄alkenyl, C₂₋₄alkynyl, halo, OR³, CN, NO₂, NHR³R³, oxo, CF₃, OCF₃, C(═O)C₁₋₄alkyl, OC(═O)C₁₋₄alkyl, or C(═O)OR³; wherein R³ is H, or C₁₋₄alkyl.

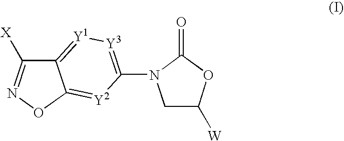

### FIELD OF INVENTION

The present invention relates to novel derivatives of benzisoxazole oxazolidinones, pharmaceutical compositions thereof, methods for their use, and methods for preparing the benzisoxazole oxazolidinone derivatives. These compounds have potent activities against gram-positive bacteria.

### BACKGROUND OF THE INVENTION

Antibacterial resistance is a global clinical and public health problem that has emerged with alarming rapidity in recent years and undoubtedly will increase in the near future. Resistance is a problem in the community as well as in health care settings, where transmission of bacteria is greatly amplified. Because multiple drug resistance is a growing problem, physicians are now confronted with infections for which there is no effective therapy. As result, structurally novel antibacterials with a new mode of action have become increasingly important in the treatment of bacterial infections.

Among newer antibacterial agents, oxazolidinone compounds are the most recent synthetic class of antimicrobials. This invention provides novel benzisoxazol oxazolidinone derivatives, which are active against a number of human and veterinary pathogens, including multiple resistant strains of bacteria.

### INFORMATION DISCLOSURE

U.S. Pat. No. 5,182,403; WO 1996/38444; DE19514313; EP785201; DE19604223; WO 2000/29409; WO 1998/54161; WO 1993/08179; and JP07309850 disclose oxazolidinones as antibacterial agents

### SUMMARY OF THE INVENTION

The present invention provides a compound of formula I

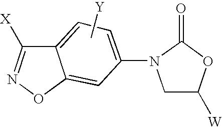

or a pharmaceutically acceptable salt thereof wherein:

### W is

(a) CH₂NHC(═Z)R¹,

(b) C(═Z)NHR², or

(c) CH₂het;

X is H, C₁₋₆alkyl, or C₂₋₆alkenyl;

### Y is H, or F;

### Z is O, or S;

### R¹ is

(a) C₁₋₆alkyl,

(b) NHC₁₋₆alkyl,

(c) C₃₋₇cycloalkyl,

(d) C₂₋₆alkenyl, or

(e) OC₁₋₄alkyl;

### R² is

(a) H,

(b) C₁₋₄alkyl, or

(c) —OC₁₋₄alkyl; and

het is a five-(5) or six-(6) membered heterocyclic ring having 1-4 heteroatoms selected from the group consisting of oxygen, sulfur, and nitrogen within the ring, wherein each carbon atom in het is optionally substituted with C₁₋₄alkyl, C₂₋₄alkenyl, C₂₋₄alkynyl, halo, OR³, CN, NO₂, NHR³R³, oxo, CF₃, OCF₃, C(═O)C₁₋₄alkyl, OC(═O)C₁₋₄alkyl, or C(═O)OR³.

In another aspect, the present invention also provides:

a pharmaceutical composition which comprises a pharmaceutically acceptable carrier and an effective amount of a compound of formula I,

a method for treating gram-positive microbial infections in a mammal by administering to the subject in need a therapeutically effective amount of a compound of formula I or a pharmaceutically acceptable salt thereof, and

a use of a compound of formula I or a pharmaceutically acceptable salt thereof to prepare a medicament for treating gram-positive microbial infections.

The invention may also provide novel intermediates and novel processes that are useful for preparing compounds of formula I.

### DETAILED DESCRIPTION OF THE INVENTION

Unless otherwise stated, the following terms used in the specification and claims have the meanings given below:

The carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, C₁₋₇ alkyl refers to alkyl of one to seven carbon atoms, inclusive.

The term alkyl, alkenyl or alkynyl refer to both straight and branched groups, but reference to an individual radical such as “propyl” embraces only the straight chain radical, a branched chain isomer such as “isopropyl” being specifically referred to.

The term “C₃₋₇cycloalkyl” refers to a cyclic saturated monovalent hydrocarbon group of three to seven carbon atoms, e.g., cyclopropyl, cyclohexyl, and the like.

The term “halo” refers to fluoro (F), chloro (Cl), bromo (Br), or iodo (I).

The term “het” is a five-(5) or six-(6) membered heterocyclic ring having 1-4 heteroatoms selected from the group consisting of oxygen, sulfur, and nitrogen within the ring. An examples of het includes, but are not limited to, pyrrole, imidazole, pyrazole, 1,2,3-triazole, 1,3,4-triazole, oxazole, thiazole, isoxazole, isothiazole, 1,3,4-oxadiazole, 1,3,4-thiadiazole, 1,2,3-thiadiazole, tetrazole, pyridine, pyrazine, pyrimidine, pyridazine, indolizine, isoindole, indole, dihydroindole, indazole, purine, quinolizine, isoquinoline, quinoline, phthalazine, quinoxaline, quinazoline, cinnoline, pteridine, carbazole, carboline, phenanthridine, acridine, phenanthroline, isothiazole, phenazine, isoxazole, isoxazolinone, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiadiazole, tetrazole, thiazolidine, thiophene, benzo[b]thiophene, morpholine, thiomorpholine, (also referred to as thiamorpholine,), piperidine, pyrrolidine, tetrahydrofuran, or the like. Another example of het includes, but are not limited to, pyridine, thiophene, furan, pyrazole, pyrimidine, 2-pyridyl, 3-pyridyl, 4-pyridyl, 2-pyrimidinyl, 4-pyrimidinyl, 5-pyrimidinyl, 3-pyridazinyl, 4-pyridazinyl, 3-pyrazinyl, 4-oxo-2-imidazolyl, 2-imidazolyl, 4-imidazolyl, 3-isoxaz-olyl, 4-is-oxaz-olyl, 5-isoxaz-olyl, 3-pyrazolyl, 4-pyrazolyl, 5-pyrazolyl, 2-oxazolyl, 4-oxazolyl, 4-oxo-2-oxazolyl, 5-oxazolyl, 1,2,3-oxathiazole, 1,2,3-oxadiazole, 1,2,4-oxadiazole, 1,2,5-oxadiazole, 1,3,4-oxadiazole, 2-thiazolyl, 4-thiazolyl, 5-thiazolyl, 3-isothiazole, 4-isothiazole, 5-isothiazole, 2-furanyl, 3-furanyl, 2-thienyl, 3-thienyl, 2-pyrrolyl, 3-pyrrolyl, 3-isopyrrolyl, 4-isopyrrolyl, 5-isopyrrolyl, 1,2,3,-oxathiazole-1-oxide, 1,2,4-oxadiazol-3-yl, 1,2,4-oxadiazol-5-yl, 5-oxo-1,2,4-oxadiazol-3-yl, 1,2,4-thiadiazol-3-yl, 1,2,5-thiadiazol-3-yl, 1,2,4-thiadiazol-5-yl, 3-oxo-1,2,4-thiadiazol-5-yl, 1,3,4-thiadiazol-5-yl, 2-oxo-1,3,4-thiadiazol-5-yl, 1,2,3-triazole-1-yl, 1,2,4-triazol-3-yl, 1,2,4-triazol-5-yl, tetrazole-1-yl, 1,2,3,4-tetrazol-5-yl, 5-oxazolyl, 3-isothiazolyl, 4-isothiazolyl and 5-isothiazolyl, 1,3,4,-oxadiazole, 4-oxo-2-thiazolinyl, or 5-methyl-1,3,4-thiadiazol-2-yl, thiazoledione, 1,2,3,4-thiatriazole, or 1,2,4-dithiazolone.

The term “pharmaceutically acceptable carrier” means a carrier that is useful in preparing a pharmaceutical composition that is generally safe, non-toxic and neither biologically nor otherwise undesirable, and includes a carrier that is acceptable for veterinary use as well as human pharmaceutical use. “A pharmaceutically acceptable carrier” as used in the specification and claims includes both one and more than one such carrier.

The term “mammal” refers to human or warm-blooded animals including livestock and companion animals. Livestock refers to animals suitable for human meat consumption. Examples include pigs, cattle, chickens, fish, turkeys, rabbits, etc. Companion animals refer to animals kept as pets such as dogs, cats, etc.

The term “optional” or “optionally” means that the subsequently described event or circumstance may, but need not, occur, and that the description includes instances where the event or circumstance occurs and instances in which it does not.

The term “treating” or “treatment” of a disease includes: (1) preventing the disease, i.e. causing the clinical symptoms of the disease not to develop in a mammal that may be exposed to or predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

The term “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

The term “prodrug” refers to compounds that are rapidly transformed in vivo to yield the parent compound of the above formulas, for example, by hydrolysis in blood. A thorough discussion is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

The term “leaving group” has the meaning conventionally associated with it in synthetic organic chemistry i.e., an atom or group capable of being displaced by a nucleophile and includes halogen, alkylsulfonyloxy, ester, or amino such as chloro, bromo, iodo, mesyloxy, tosyloxy, trifluorosulfonyloxy, methoxy, N,O-dimethylhydroxyl-amino, and the like.

Compounds that have the same molecular formula but differ in the nature or sequence of bonding of their atoms or the arrangement of their atoms in space are termed “isomers”. Isomers that differ in the arrangement of their atoms in space are termed “stereoisomers”.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, tautomeric, or stereoisomeric form, or mixture thereof, of a compound of the invention, which possesses the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine antiviral activity using the standard tests described herein, or using other similar tests which are well known in the art.

The compounds of the present invention are generally named according to the IUPAC or CAS nomenclature system. Abbreviations which are well known to one of ordinary skill in the art may be used (e.g. “Ph” for phenyl, “Me” for methyl, “Et” for ethyl, “h” for an hour or hours and “rt” for room temperature). Specific and preferred values listed below for radicals, substituents, and ranges, are for illustration only; they do not exclude other defined values or other values within defined ranges for the radicals and substituents.

Specifically, alkyl denotes both straight and branched groups; but reference to an individual radical such as “propyl” embraces only the straight chain radical, a branched chain isomer such as “isopropyl” being specifically referred to.

Specifically, alkyl is methyl, ethyl, propyl, isopropyl, butyl, iso-butyl, sec-butyl, and their isomeric forms thereof.

Specifically, cycloalkyl is cyclopropyl, cyclobutyl, cyclopentyl, cyclohexyl, and their isomeric forms thereof.

Specifically, halo is fluoro (F), chloro (Cl).

Specifically, Y is H.

Specifically W is CH₂NHC(═O)R¹.

Specifically, R¹ is C₁₋₄alkyl, optionally substituted with one, two or three fluoro (F), or chloro (Cl).

Specifically, R¹ is CH₃, or CH₂CH₃.

Specifically, W is CH₂het.

Specifically, W is 1,2,3-triazole-1-yl methyl.

Specifically, W is C(═O)NHR².

Specifically, R² is H

Specifically, R² is C₁₋₄alkyl.

Specifically, R² is CH₃, or CH₂CH₃.

Specifically, R² is OC₁₋₄alkyl.

Specifically, R² is OCH₃, or OCH₂CH₃.

Specifically, X is Me, Et, i-Pr, or sec-Bu.

Examples of the present invention include:
(1) (S)—N-[3-(3-methyl-benzo[d]isoxazol-6-yl)-2-oxo-oxazolidin-5-ylmethyl]-acetamide,
(2) (S)—N-[3-(3-methyl-benzo[d]isoxazol-6-yl)-2-oxo-oxazolidin-5-ylmethyl]-propionamide,
(3) (S)-[3-(3-methyl-benzo[d]isoxazol-6-yl)-2-oxo-oxazolidin-5-ylmethyl]-carbamic acid methyl ester,
(4) (R)-3-(3-methyl-benzo[d]isoxazol-6-yl)-5-[1,2,3]triazol-1-ylmethyl-oxazolidin-2-one,
(5) (R)-3-(3-methyl-benzo[d]isoxazol-6-yl)-5-(4-trimethylsilanylethynyl-[1,2,3]triazol-1-ylmethyl)-oxazolidin-2-one,
(6) (R)-3-(3-methyl-benzo[d]isoxazol-6-yl)-2-oxo-oxazolidine-5-carboxylic acid amide,
(7) (R)-3-(3-methyl-benzo[d]isoxazol-6-yl)-2-oxo-oxazolidine-5-carboxylic acid methylamide,
(8) (R)-3-(3-methyl-benzo[d]isoxazol-6-yl)-2-oxo-oxazolidine-5-carboxylic acid methoxy-amide, or
(9) R)-3-(3-methyl-benzo[d]isoxazol-6-yl)-2-oxo-oxazolidine-5-carboxylic acid ethoxy-amide.

Compounds of this invention can be prepared in accordance with one or more of the Schemes discussed below. All of the starting materials are either commercially available or can be prepared by procedures that would be well known to one of ordinary skill in organic chemistry. The variables used in the Schemes are as defined below, or as in the summary of the invention or claims.

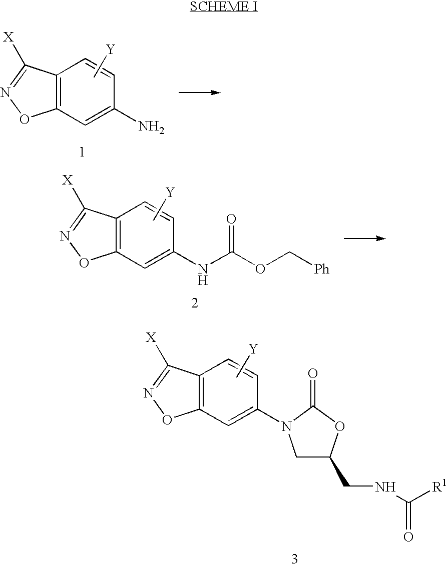

As shown in Scheme I, the 3-substituted-6-amino-1,2-benzisoxazole 1 can be reacted with a chloroformate, such as benzyl chloroformate, using methods known to one skilled in the art. Carbamate 2 can be treated with a base, such as lithium tert-butoxide, and (S)—N-[2-(acetyloxy)-3-chloropropyl]acetamide to give oxazolidinone 3.

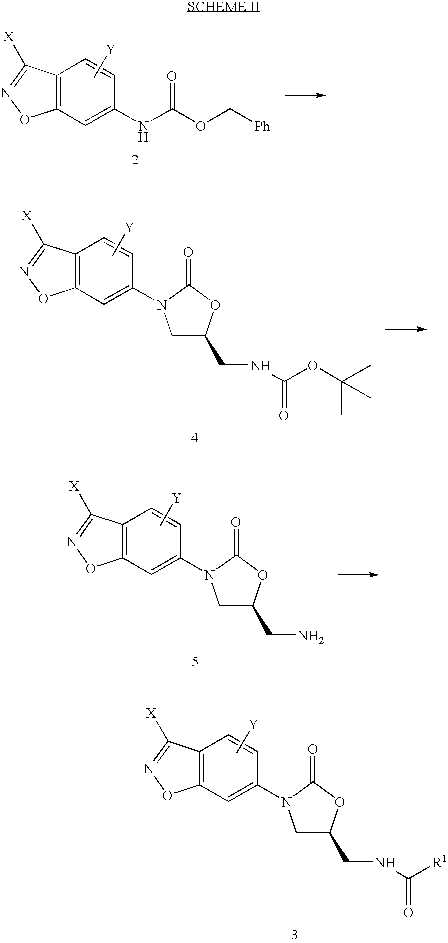

Carbamate 2 can also be reacted with a base, such as lithium tert-butoxide, and treated with tert-butyl-(2S)-3-chloro-2-hydroxypropylcarbamate to give oxazolidinone 4 (Scheme II). The tert-butoxycarbonyl group of compound 4 can be cleaved with an acid, such as hydrochloric acid, to give oxazolidinone 5. Subsequent treatment of oxazolidinone 5 with various known acylating reagents, such as propionic anhydride, or chloroformate reagents, such as methyl chloroformate, will give oxazolidinones such as 3.

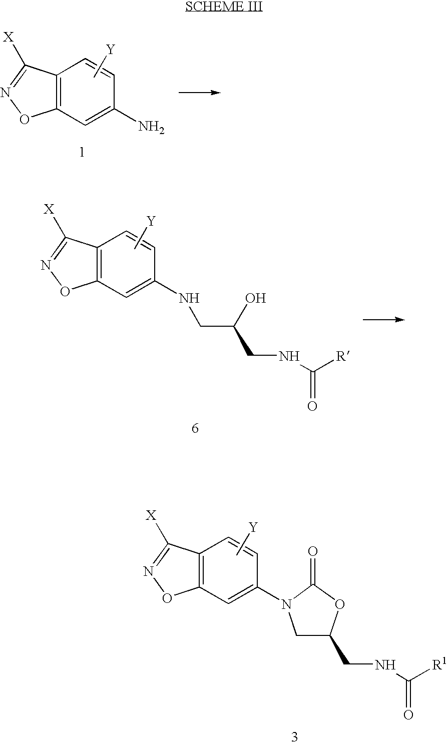

Alternatively, the 3-substituted-6-amino-1,2-benzisoxazole 1 can be reacted with appropriately substituted epoxides, such as (S)-oxiranylmethyl acetamide, in the presence of a Lewis acid, such as lithium trifluoromethanesulfonate, to give compounds such as 6 (Scheme III). Ring closure to form the oxazolidinone 3 can be achieved with various methods known to one skilled in the art, such as treatment of compound 6 with 1,1′-carbonyldiimidazole.

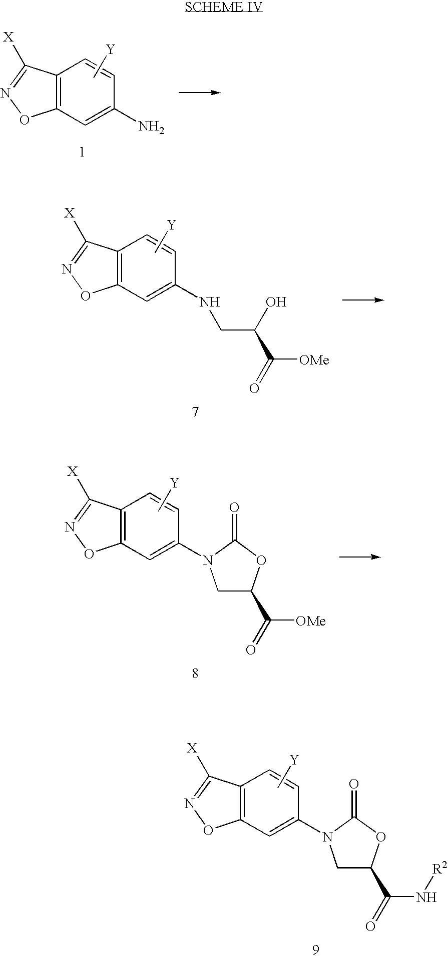

As shown in Scheme IV, 3-substituted-6-amino-1,2-benzisoxazole 1 can be reacted with methyl (2R)-glycidate in the presence of a Lewis acid, such as lithium trifluoromethanesulfonate, in a solvent such as acetonitrile, to give compound 7. Ring closure of compound 7 to the oxazolidinone 8 can be achieved using various methods known to those skilled in the art, including use of 1,1′-carbonyldiimidazole. Conversion of ester 8 to amide 9 may be achieved by treatment with ammonia or by treatment with various substituted amines, such as methylamine.

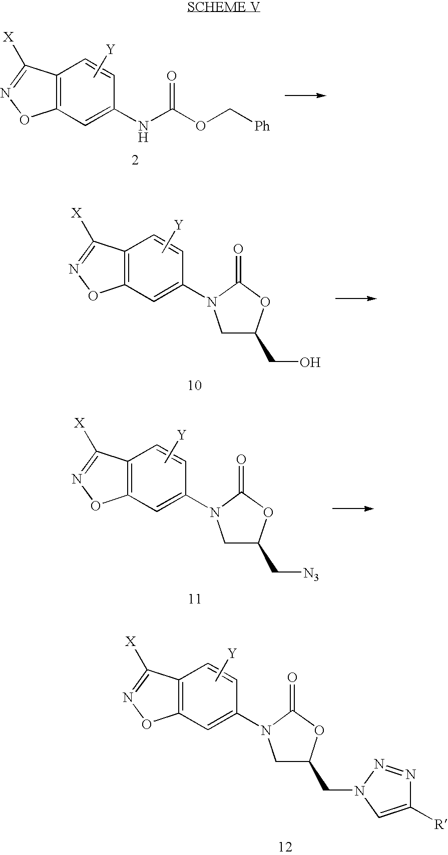

Carbamate 2 can be converted to oxazolidinone 10 by treatment with a base, such as n-butyllithium, followed by addition of (R)-(−)-glycidyl butyrate (Scheme V). The primary alcohol of compound 10 can be converted to azide 11 using methods known to those skilled in the art. For example, alcohol 10 can be converted to the mesylate by treatment with methanesulfonyl chloride, and the mesylate can then be converted to the azide 11 by treatment with sodium azide. The azide 11 can be converted to a triazole or substituted triazole 12 using methods known to those skilled in the art. For example, azide 11 can be reacted with 2,5-norbornadiene to give triazole 12 (R′═H).

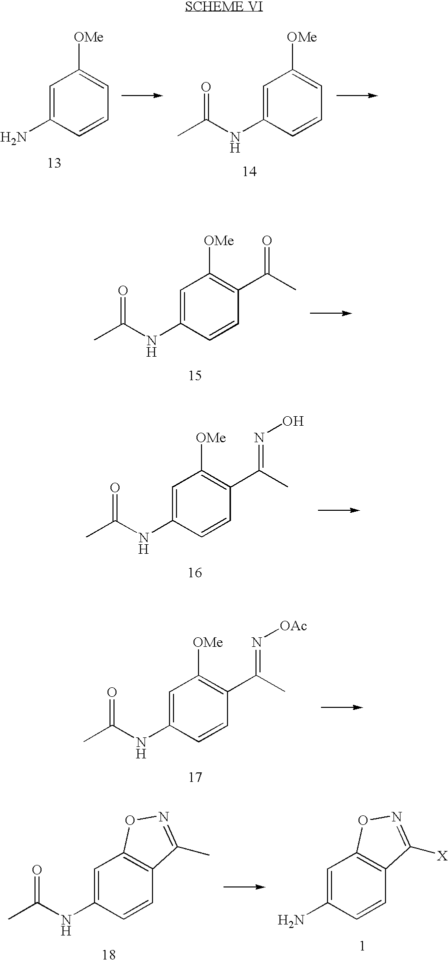

The requisite benzisoxazole is most conveniently prepared as shown in Scheme VI. The first step is acylation of appropriate aniline (13) with acetic anhydride to yield the corresponding acetamides (14), which on acylation with acid chloride in the presence of a Lewis acid (e.g., aluminum chloride) to afford the acylated analogs (15). Reaction of ketone (15) with hydroxylamine in the presence of sodium acetate provides oxime (16), which is acylated (17), followed by refluxing with a base like pyridine provided the ring closure material, benzisoxazole (18). Heating (18) under acidic conditions undergoes deprotection to afford aniline (1).

### Medical and Veterinary Uses

The compound of the present invention may be used for the treatment of infectious, Gram-positive bacterial infections caused by a variety of bacterial organisms, including those that require long-term therapy (>28 days).

Examples of the bacterial organisms include gram-positive bacteria such as multiple resistant staphylococci, for example S. aureus and S. epidermidis; multiple resistant streptococci, for example S. pneumoniae and S. pyogenes; and multiple resistant Enterococci, for example E. faecalis, gram negative aerobic bacteria such as Haemophilus, for example H. influenzae and Moraxella, for example M. catarrhalis; as well as anaerobic organisms such as bacteroides and clostridia species, and acid-fast organisms such as Mycobacteria, for example M. tuberculosis, and/or Mycobacterium avium. Other examples include Escherichia, for example E. coli. intercellular microbes, for example Chlamydia and Rickettsiae.

Examples of infections that may be treated with the compound of the present invention include central nervous system infections, external ear infections, infections of the middle ear, such as acute otitis media, infections of the cranial sinuses, eye infections, infections of the oral cavity, such as infections of the teeth, gums and mucosa, upper respiratory tract infections, lower respiratory tract infections, genitourinary infections, gastrointestinal infections, gynecological infections, septicemia, bone and joint infections, skin and skin structure infections, bacterial endocarditis, burns, antibacterial prophylaxis of surgery, and antibacterial prophylaxis in immunosuppressed patients, such as patients receiving cancer chemotherapy, or organ transplant patients. Specifically, infectious diseases that may be treated with the compound of the present invention are gram-positive infections such as osteomyelitis, endocarditis and diabetic foot.

### Antibacterial Activity

The in vitro antibacterial activity of the compounds of the present invention may be assessed by following procedures recommended in (1) National Committee for Clinical Laboratory Standards (January 2003), Methods for dilution antimicrobial tests for bacteria that grow aerobically, Approved Standard (6^{th} ed), M7-A6, NCCLS, Wayne, Pa.; (2) National Committee for Clinical Laboratory Standards (March 2001), Methods for antimicrobial susceptibility testing of anaerobic bacteria, Approved Standard (5^{th} ed), M11-A4, NCCLS, Wayne, Pa.; (3) National Committee for Clinical Laboratory Standards (Jan. 2003), MIC testing supplemental tables, M100-S13 (for use with M7-A6), NCCLS, Wayne, Pa.; and (4) Murray P R, Baron E J, Jorgensen J H, et al. Manual of Clinical Microbiology (8^{th} ed) Washington, D.C.: American Society for Microbiology Press, 2003. The antibacterial activity can be presented in the form of MIC value. The MIC value is the lowest concentration of drug, which prevented macroscopically visible growth under the conditions of the test. Table 1 lists the in vitro antibacterial activity of the present invention.

TABLE 1
Results of in vitro antibacterial activity MIC₅ (μg/mL)

| | S. aureus | S. pneumoniae | E. faecalis |
|---|---|---|---|
| Example No. | UC-76 SA-1 | SV1 SP-3 | MGH-2 EF 1-1 |
| 1 | 2 | 1 | 2 |
| 2 | 2 | 2 | 4 |
| 3 | 4 | 2 | 4 |
| 4 | 4 | 2 | 4 |
| 5 | 8 | 4 | 4 |
| 6 | 4 | 4 | 8 |
| 7 | 4 | 4 | 4 |
| 8 | 4 | 8 | 4 |
| | 4 | 8 | 4 |

### Pharmaceutical Salts

The compound of formula I may be used in its native form or as a salt. In cases where forming a stable nontoxic acid or base salt is desired, administration of the compound as a pharmaceutically acceptable salt may be appropriate. Examples of pharmaceutically acceptable salts of the present invention include inorganic salts such as hydrochloride, hydrobromide, sulfate, nitrate, bicarbonate, carbonate salts, and organic salts such as tosylate, methanesulfonate, acetate, citrate, malonate, tartarate, succinate, benzoate, ascorbate, etoglutarate, and glycerophosphate. Pharmaceutically acceptable salts may be obtained using standard procedures well known in the art, for example, reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

### Routes of Administration

In therapeutic use for treating, or combating, bacterial infections in a mammal (i.e. human and animals), a compound of the present invention or its pharmaceutical compositions can be administered orally, parenterally, topically, rectally, transmucosally, or intestinally.

Parenteral administrations include indirect injections to generate a systemic effect or direct injections to the afflicted area. Examples of parenteral administrations are subcutaneous, intravenous, intramuscular, intradermal, intrathecal, intraocular, intranasal, intraventricular injections or infusions techniques.

Topical administrations include the treatment of infectious areas or organs readily accessibly by local application, such as, for example, eyes, ears including external and middle ear infections, vaginal, open wound, skins including the surface skin and the underneath dermal structures, or other lower intestinal tract. It also includes transdermal delivery to generate a systemic effect.

The rectal administration includes the form of suppositories.

The transmucosal administration includes nasal aerosol or inhalation applications.

The preferred routes of administration are oral and parenteral.

### Composition/Formulation

Pharmaceutical compositions of the present invention may be manufactured by processes well known in the art, e.g., by means of conventional mixing, dissolving, granulation, dragee-making, levigating, emulsifying, encapsulating, entrapping, lyophilizing processes or spray drying.

Pharmaceutical compositions for use in accordance with the present invention may be formulated in conventional manner using one or more physiologically acceptable carriers comprising excipients and auxiliaries, which facilitate processing of the active compound into preparations, which can be used pharmaceutically. Proper formulation is dependent upon the route of administration chosen.

For oral administration, the compound can be formulated by combining the active compound with pharmaceutically acceptable carriers well known in the art. Such carriers enable the compound of the invention to be formulated as tablets, pills, lozenges, dragees, capsules, liquids, solutions, emulsions, gels, syrups, slurries, suspensions and the like, for oral ingestion by a patient. A carrier can be at least one substance which may also function as a diluent, flavoring agent, solubilizer, lubricant, suspending agent, binder, tablet disintegrating agent, and encapsulating agent. Examples of such carriers or excipients include, but are not limited to, magnesium carbonate, magnesium stearate, talc, sugar, lactose, sucrose, pectin, dextrin, mannitol, sorbitol, starches, gelatin, cellulosic materials, low melting wax, cocoa butter or powder, polymers such as polyethylene glycols and other pharmaceutical acceptable materials.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compound may be dissolved or suspended in suitable liquids, such as fatty oils, liquid paraffin, liquid polyethylene glycols, cremophor, capmul, medium or long chain mono-, di- or triglycerides. Stabilizers may be added in these formulations, also.

Liquid form compositions include solutions, suspensions and emulsions. For example, there may be provided solutions of the compound of this invention dissolved in water and water-propylene glycol and water-polyethylene glycol systems, optionally containing suitable conventional coloring agents, flavoring agents, stabilizers and thickening agents.

The compound may also be formulated for parenteral administration, e.g., by injections, bolus injection or continuous infusion. Formulations for parenteral administration may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

For injection, the compound of the invention may be formulated in aqueous solution, preferably in physiologically compatible buffers or physiological saline buffer. Suitable buffering agents include trisodium orthophosphate, sodium bicarbonate, sodium citrate, N-methylglucamine, L(+)-lysine and L(+)-arginine.

Parenteral administrations also include aqueous solutions of a water soluble form, such as, without limitation, a salt, of the active compound. Additionally, suspensions of the active compound may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances, which increase the viscosity of the suspension, such as sodium carboxymethyl cellulose, sorbitol, or dextran. Optionally, the suspension may also contain suitable stabilizers and/or agents that increase the solubility of the compound to allow for the preparation of highly concentrated solutions.

Alternatively, the active ingredient may be in powder form for constitution with a suitable vehicle, e.g., sterile, pyrogen-free water, before use.

For suppository administration, the compound may also be formulated by mixing the agent with a suitable non-irritating excipient, which is solid at room temperature but liquid at rectal temperature and therefore will melt in the rectum to release the drug. Such materials include cocoa butter, beeswax and other glycerides.

For administration by inhalation, compound of the present invention can be conveniently delivered through an aerosol spray in the form of solution, dry powder, or suspensions. The aerosol may use a pressurized pack or a nebulizer and a suitable propellant. In the case of a pressurized aerosol, the dosage unit may be controlled by providing a valve to deliver a metered amount. Capsules and cartridges of, for example, gelatin for use in an inhaler may be formulated containing a power base such as lactose or starch.

For topical applications, the pharmaceutical composition may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion such as suspensions, emulsion, or cream containing the active components suspended or dissolved in one or more pharmaceutically acceptable carriers. Suitable carriers include, but are not limited to, mineral oil, sorbitan monosterate, polysorbate 60, cetyl esters wax, cetearyl alcohol, 2-octyldodecanol, benzyl alcohol and water.

For ophthalmic and otitis uses, the pharmaceutical compositions may be formulated as micronized suspensions in isotonic, pH adjusted sterile saline, or preferably, as solutions in isotonic, pH adjusted sterile saline, either with or without a preservative such as a benzylalkonium chloride. Alternatively, for ophthalmic uses, the pharmaceutical compositions may be formulated in an ointment such as petrolatum.

In addition to the formulations described previously, the compound may also be formulated as depot preparations. Such long acting formulations may be in the form of implants. A compound of this invention may be formulated for this route of administration with suitable polymers, hydrophobic materials, or as a sparing soluble derivative such as, without limitation, a sparingly soluble salt.

Additionally, the compound may be delivered using a sustained-release system. Various sustained-release materials have been established and are well known by those skilled in the art. Sustained-release capsules may, depending on their chemical nature, release the compound for 24 hours or for up to several days.

### Dosage

Pharmaceutical compositions suitable for use in the present invention include compositions wherein the active ingredients are contained in an amount sufficient to achieve the intended purpose, i.e., the treatment or prevent of infectious diseases. More specifically, a therapeutically effective amount means an amount of compound effective to prevent, alleviate or ameliorate symptoms of disease or prolong the survival of the subject being treated.

The quantity of active component, that is the compound of this invention, in the pharmaceutical composition and unit dosage form thereof may be varied or adjusted widely depending upon the manner of administration, the potency of the particular compound and the desired concentration. Determination of a therapeutically effective amount is well within the capability of those skilled in the art. Generally, the quantity of active component will range between 0.5% to 90% by weight of the composition.

Generally, a therapeutically effective amount of dosage of active component will be in the range of about 0.1 to about 400 mg/kg of body weight/day, more preferably about 1.0 to about 50 mg/kg of body weight/day. It is to be understood that the dosages may vary depending upon the requirements of each subject and the severity of the bacterial infection being treated. In average, the effective amount of active component is about 200 mg to 800 mg and preferable 600 mg per day.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Also, it is to be understood that the initial dosage administered may be increased beyond the above upper level in order to rapidly achieve the desired plasma concentration. On the other hand, the initial dosage may be smaller than the optimum and the daily dosage may be progressively increased during the course of treatment depending on the particular situation. If desired, the daily dose may also be divided into multiple doses for administration, e.g., two to four times per day.

In cases of local administration or selective uptake, the effective local concentration of the drug may not be related to plasma concentration and other procedures know in the art may be used to determine the desired dosage amount.

### EXAMPLES

The compounds of this invention can be prepared in accordance with the examples discussed below. All of the starting materials are either commercially available or can be prepared by procedures that would be well known to one of ordinary skill in organic chemistry. Also, in the discussion the preparations below, the following abbreviations have the following meanings. If an abbreviation is not defined, it has its generally accepted meaning.
bm=broad multiplet
BOC=tert-butoxycarbonyl
bd=broad doublet
bs=broad singlet
CDI=1,1O-carbodiimidazole
d=doublet
dd=doublet of doublets
dq=doublet of quartets
dt=doublet of triplets
DMF=dimethylformamide
DMAP=dimethylaminopyridine
DMSO=dimethyl sulfoxide
eq.=equivalents
g=grams
h=hours
HPLC=high pressure liquid chromatography
HATU=N-[(dimethylamino)-1H-1,2,3-triazolo-[4,5-b]pyridin-1-yl-methylene]-N-methylmethanaminium hexafluorophosphate N-oxide
LG=leaving group
m=multiplet
M=molar
M %=mole percent
max=maximum
meq=milliequivalent
mg=milligram
mL=milliliter
mm=millimeter
mmol=millimol
q=quartet
s=singlet
t or tr=triplet
TBS=tributylsilyl
TFA=trifluoroacetic acid
THF=tetrahydrofuran
TLC=thin layer chromatography
p-TLC=preparative thin layer chromatography
μL=microliter
N=normality
MeOH=methanol
DCM=dichloromethane
HCl=hydrochloric acid
ACN=acetonitrile
MS=mass spectrometry
rt=room temperature
EtOAc=ethyl acetate
EtO=ethoxy
Ac=acetate
NMP=1-methyl-2-pyrrolidinone
μL=microliter
J=coupling constant
NMR=Nuclear magnetic resonance
MHz=megahertz
Hz=hertz
m/z=mass to charge ratio
min=minutes
Boc=tert-butoxycarbonyl
CBZ=benzyloxycarbonyl
DCC=1,3-dicyclohexylcarbodiimide
PyBop=benzotriazole-1-yl-oxy-trispyrrolidinophosphonium hexafluorophosphate

### Example 1 Preparation of (S)—N-[3-(3-methyl-benzo[d]isoxazol-6-yl)-2-oxo-oxazolidin-5-ylmethyl]-acetamide

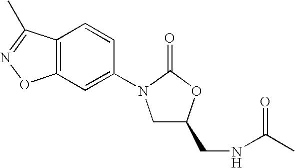

### Step 1: Preparation of 3-methoxyacetanilide

3-methoxyacetanilide is prepared according to Akhavan-Tafti, H.; et al. J. Org. Chem. 1998, 63, 930-937: Acetic anhydride (20 mL; 212.0 mmol) is added to a mixture of 3-methoxyaniline (1) (20 g, 162.4 mmol) in 20 mL of acetic acid at 0° C. The reaction is stirred overnight at RT and then poured into 100 g of ice in 100 mL of water. The resultant solid is collected by filtration (24.9 g, 93% yield) and used without further purification. ¹H NMR (400 MHz, CDCl₃): δ 7.48 (br s, 1H), 7.25 (m, 1H), 7.17 (t, J=8.0 Hz, 1H), 6.95 (dd, J=8.0, 1.0 Hz, 1H), 6.63 (dd, J=8.0, 1.8 Hz, 1H), 3.76 (s, 3H), 2.13 (s, 3H).

### Step 2: Preparation of 4-acetamido-2-hydroxyacetophenone

The title compound is prepared based on a modified procedure of Elliott, J. M.; et al. J. Med. Chem., 1992, 35, 3973-3976: Aluminum chloride (27 g, 206 mmol) is added in portions over 20 min to a mechanically stirred solution of 3-methoxyacetanilinde (10 g, 60.5 mmol), acetyl chloride (12.5 mL, 175.6 mmol), and 25 mL of CH₂Cl₂. After all of the dichloromethane is removed from the reaction mixture by distillation, the resultant viscous mixture is heated (without stirring) at 80° C. for 3.5 h. Chlorobenzene (60 mL) is then added and the mixture is heated to reflux (132° C.) and stirred at reflux for 1 h. The resultant syrup is cooled to 0° C. and crushed ice is added slowly while stirring. The resultant solid is collected by filtration and purified by silica gel chromatography to afford 7.34 g (63%) of the title compound. ¹H NMR (400 MHz, DMSO-d₆): δ 12.31 (s, 1H), 10.26 (s, 1H), 7.83 (d, J=8.8 Hz, 1H), 7.34 (d, J=1.8 Hz, 1H), 7.05 (dd, J=8.8, 1.8 Hz, 1H), 2.56 (s, 3H), 2.08 (s, 3H). MS-APCI (m/z+): 194 (M+1).

### Step 3: Preparation of 6-acetamido-3-methyl-1,2-benzisoxazole

The title compound is prepared according to Villalobos, A.; et al. J. Med. Chem. 1994, 37, 2721-2734: A solution of hydroxylamine hydrochloride (4.18 g, 60.1 mmol) and sodium acetate trihydrate (8.38 g, 61.6 mmol) in 70 mL of 7:3 EtOH:H₂O is added to a slurry of 4-acetamido-2-hydroxyacetophenone (7.00 g, 36.2 mmol) in 50 mL of 7:3 EtOH:H₂O. The mixture is heated to reflux for 4 h and then stirred at RT overnight. The resultant solid is collected by filtration and washed with water to give 4.55 g (60%) of the oxime, which is used without further purification. ¹H NMR (400 MHz, DMSO-d₆): δ 11.67 (br s, 1H), 11.37 (s, 1H), 9.97 (s, 1H), 739 (d, J=8.6 Hz, 1H), 7.24 (d, J=2.0 Hz, 1H), 7.04 (dd, J=8.6, 2.0 Hz, 1H), 2.21 (s, 3H), 2.03 (s, 3H). MS-APCI (m/z+): 209 (M+1), 191.

Acetic anhydride (11.4 mL, 120.4 mmol) is added to the oxime (4.5 g, 21.6 mmol) and the resultant slurry is heated to 130° C. until a clear solution formed (5 min). After cooling to RT, the resultant solid is collected by filtration and washed with water. The solid is slurried in 1:1 CH₂Cl₂:hexanes and filtered to give 3.59 g (64%) of the oxime acetate as a solid. ¹H NMR (400 MHz, DMSO-d₆): δ 11.00 (s, 1H), 10.07 (s, 1H), 7.46 (d, J=8.4 Hz, 1H), 7.36 (s, 1H), 7.06 (dd, J=8.4, 1.2 Hz, 1H), 2.38 (s, 3H), 2.22 (s, 3H), 2.05 (s, 3H). MS-APCI (m/z+): 251 (M+1), 191. Anal. Calcd for C₁₂H₁₄N₂O₄: C, 57.59; H, 5.64; N, 11.19. Found: C, 57.58; H, 5.55; N, 11.13.

A slurry of pyridine (31 mL) and the oxime acetate (3.49 g, 13.94 mmol) is heated to reflux (125° C.) overnight. The resultant clear solution is cooled to RT and poured into 1 M HCl. The aq layer is extracted with EtOAc and the organic layer is washed with 1 M HCl, water and brine. The aq layer is back-extracted with EtOAc and the combined org layers are dried over Na₂SO₄ and conc in vacuo. The crude material is stirred with 50% EtOAc in hexanes overnight and the resultant solid is collected by filtration and purified by silica gel chromatography to give 1.76 g (66%) of 6-Acetamido-3-methyl-1,2-benzisoxazole as a solid. ¹H NMR (400 MHz, DMSO-d₆): δ 10.35 (s, 1H), 8.14 (s, 1H), 7.74 (d, J=8.6 Hz, 1H), 7.36 (d, J=8.6 Hz, 1H), 2.50 (s, 3H), 2.11 (s, 3H). ¹³C NMR (125.7 MHz, DMSO): δ 169.0, 162.8, 154.7, 141.4, 121.9, 117.0, 115.6, 98.3, 24.2, 9.5. MS-APCI (m/z+): 191 (M+1).

### Step 4: Preparation of 6-amino-3-methyl-1,2-benzisoxazole

The title compound is prepared according to Villalobos, A.; et al. J. Med. Chem. 1994, 37, 2721-2734: A mixture of 6-acetamido-3-methyl-1,2-benzisoxazole (0.365 g, 1.92 mmol) and 1 M aq HCl (7 mL) is heated to reflux for 45 thin at which point a clear solution is obtained. An additional 2 mL of 1 M aq HCl is added and reflux is continued until the reaction is complete (about 2 h). After cooling to RT overnight, the solution is made basic (pH ˜8) by addition of 10% aq NaOH. The resultant solid is collected by filtration and washed with water to give 0.171 g (60%) of the title compound. ¹H NMR (400 MHz, CDCl₃): δ 7.32 (d, J=8.4 Hz, 1H), 6.69 (d, J=1.6 Hz, 1H), 6.61 (dd, J=8.4, 1.6 Hz, 1H), 4.01 (br s, 2H), 2.46 (s, 3H). MS-APCI (m/z+): 149 (M+1), 190 (M+1+CH₃CN).

### Step 5: Preparation of (3-methyl-benzo[d]isoxazole-6-yl)-carbamic acid benzyl ester

6-Amino-3-methyl-1,2-benzisoxazole (1.12 g, 7.56 mmol), THF (24 mL), H₂O (12 mL), NaHCO₃ (2.5 g, 30 mmol), and benzyl chloroformate (2.4 mL, 16.63 mmol) are stirred at RT overnight. Ethyl acetate and water are added and the layers are separated. The org layer is washed with water, brine, dried over Na₂SO₄ and conc in vacuo. Purification by silica gel chromatography afforded 2.12 g (99%) of the title compound. ¹H NMR (400 MHz, DMSO-d₆): δ 10.26 (s, 1H), 7.92 (d, J=1.2 Hz, 1H), 7.73 (d, J=8.4 Hz, 1H), 7.40 (m, 6H), 5.21 (s, 2H), 2.50 (s, 3H). MS-APCI (m/z+): 283 (M+1).

### Step 6: Preparation of (S)-5 N-[3-(3-methyl-benzo[d]isoxazol-6-yl)-2-oxo-oxazolidin-5-ylmethyl]-acetamide

Lithium tert-butoxide (1.0 M in hexanes, 3.0 mL, 3.0 mmol) is added dropwise to a solution of (3-methyl-benzo[d]isoxazol-6-yl)-carbamic acid benzyl ester (0.282 g, 1.0 mmol) in methanol (81 μL, 2.0 mmol) and DMF (1.0 mL). After cooling to 0° C., (S)—N-[2-(acetyloxy)-3-chloropropyl]acetamide (0.387 g, 2.0 mmol) is added in one portion, the ice bath is removed and the mixture is stirred at RT overnight. The reaction is quenched with satd NH₄Cl and the aq layer is extracted with CH₂Cl₂ twice. The combined org layer is washed with water, brine, dried over Na₂SO₄ and conc in vacuo. Purification by silica gel chromatography afforded 0.129 g (45%) of the title compound as a solid. ¹H NMR (400 MHz, CDCl₃): δ 7.68 (m, 1H), 7.57 (m, 2H), 6.02 (m, 1H), 4.81 (m, 1H), 4.13 (t, J=9.0 Hz, 1H), 3.87 (dd, J=9.0, 6.6 Hz, 1H), 3.72 (ddd, J=15.0, 6.0, 3.2 Hz, 1H), 3.64 (dt, J=14.4, 6.0 Hz, 1H), 2.55 (s, 3H), 2.01 (s, 3H). MS-APCI (m/z+): 290 (M+1), 246.

### Preparation I (S)-[3-(3-Methyl-benzo[d]isoxazol-6-yl)-2-oxo-oxazolidin-5-ylmethyl]-carbamic acid tert-butyl ester

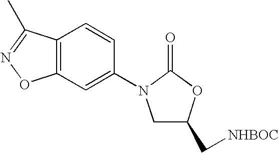

Lithium tert-butoxide (1.0 M in hexanes, 9.0 mL, 9.0 mmol) is added dropwise to a 0° C. solution of (3-methyl-benzo[d]isoxazol-6-yl)-carbamic acid benzyl ester (0.847 g, 3.0 mmol) and tert-butyl-2S)-3-chloro-2-hydroxypropylcarbamate (0.944 g, 4.50 mmol) in DMF (4.5 mL). The reaction is allowed to warm to RT and is stirred for 72 h. The reaction is quenched with satd NH₄Cl and the aq layer is extracted with CH₂Cl₂. The org layer is washed with water, brine, dried over Na₂SO₄ and conc in vacuo. Purification by silica gel chromatography afforded 0.54 g (52%) of the title compound. ¹H NMR (400 MHz, CDCl₃): δ 7.61 (m, 3H), 4.98 (br s, 1H), 4.78 (m, 1H), 4.10 (t, J=8.8 Hz, 1H), 3.93 (dd, J=9.2, 6.4 Hz, 1H), 3.54 (m, 2H), 2.54 (s, 3H), 1.37 (s, 9H). MS-APCI (m/z+): 347, 248.

### Preparation II (S)-Aminomethyl-3-(3-methyl-benzo[d]isoxazol-6-yl)-oxazolidin-2-one hydrochloride

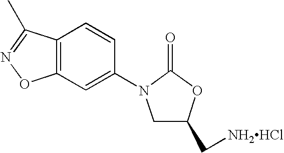

A 4M solution of HCl in dioxane (4.6 mL, 18 mmol) is added to a solution of [3-(3-Methyl-benzo[d]isoxazol-6-yl)-2-oxo-oxazolidin-5(S)-ylmethyl]-carbamic acid tert-butyl ester (0.425 g, 1.22 mmol) and anisole (0.1 mL) in THF (12 mL). After stirring at RT overnight, an additional 10 mL of 4 M HCl in dioxane is added and the mixture is again stirred overnight. The reaction mixture is diluted with ether and the reaction volume is reduced (by ˜½) using a continuous stream of N₂. The resultant solid is collected by filtration and washed with ether to give 0.307 g (88%) of the title compound which is used without further purification. ¹H NMR (400 MHz, DMSO-d₆): δ 8.33 (br s, 3H), 7.88 (d, J=8.8 Hz, 1H), 7.82 (d, J=1.4 Hz, 1H), 7.67 (dd, J=8.8, 1.4 Hz, 1H), 5.00 (m, 1H), 4.30 (t, J=9.4 Hz, 1H), 3.99 (dd, J=8.8, 6.8 Hz, 1H), 3.27 (m, 2H), 2.54 (s, 3H). MS-APCI (m/z+): 248 (M+1).

### Example 2 Preparation of (S)—N-[3-(3-methyl-benzo[d]isoxazol-6-yl)-2-oxo-oxazolidin-5-ylmethyl]-propionamide

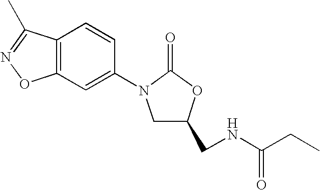

A solution of 5(S)-aminomethyl-3-(3-methyl-benzo[d]isoxazol-6-yl)-oxazolidin-2-one hydrochloride (0.13 g, 0.458 mmol), THF (2 mL), H₂O (1 mL), NaHCO₃ (0.12 g, 1.40 mmol), and propionic anhydride (88 μL, 0.687 mmol) are stirred at RT for 2 h. The mixture is diluted with CH₂Cl₂ and the layers are separated. The org layer is washed with water (3×), brine, dried over Na₂SO₄, conc in vacuo and then dried under vacuum (40° C.) to give 0.133 g (96%) of the title compound as a solid. ¹H NMR (400 MHz, DMSO-d₆): δ 8.19 (t, J=5.6 Hz, 1H), 7.85 (d, J=8.6 Hz, 1H), 7.81 (d, J=2.0 Hz, 1H), 7.67 (dd, J=8.6, 2.0 Hz, 1H), 4.78 (m, 1H), 4.22 (t, J=9.2 Hz, 1H), 3.86 (dd, J=9.2, 6.4 Hz, 1H), 3.45 (m, 2H), 2.53 (s, 3H), 2.09 (q, J=7.6 Hz, 2H), 0.94 (t, J=7.6 Hz, 3H). MS-APCI (m/z+): 304 (M+1), 260.

### Example 3 Preparation of (S)-[3-(3-methyl-benzo[d]isoxazol-6-yl)-2-oxo-oxazolidin-5-ylmethyl]-carbamic acid methyl ester

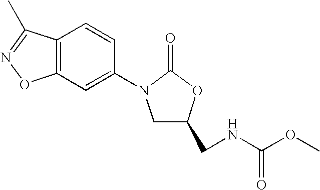

A solution of 5(S)-aminomethyl-3-(3-methyl-benzo[d]isoxazol-6-yl)-oxazolidin-2-one hydrochloride (0.13 g, 0.458 mmol), THF (2 mL), H₂O (1 mL), NaHCO₃ (0.12 g, 1.40 mmol), and methyl chloroformate (53 μL, 0.687 mmol) are stirred at RT for 2 h. The mixture is diluted with CH₂Cl₂ and the layers are separated. The org layer is washed with water (3×), brine, dried over Na₂SO₄, conc in vacuo, and then dried under vacuum (40° C.) to give 0.139 g (99%) of the title compound as a solid. ¹H NMR (400 MHz, DMSO-d₆): δ 7.85 (d, J=8.8 Hz, 1H), 7.81 (d, J=1.2 Hz, 1H), 7.68 (dd, J=8.8, 1.2 Hz, 1H), 7.55 (m, 1H), 4.77 (m, 1H), 4.23 (t, J=8.8 Hz, 1H), 3.88 (dd, J=8.8, 6.4 Hz, 1H), 3.54 (s, 3H), 3.38 (m, 2H), 2.54 (s, 3H). MS-APCI (m/z+): 306 (M+1), 248.

### Preparation III (R)-Hydroxymethyl-3-(3-methyl-benzo[d]isoxazol-6-yl)-oxazolidin-2-one

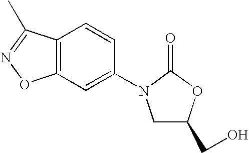

A solution of (3-methyl-benzo[d]isoxazol-6-yl)-carbamic acid benzyl ester (0.827 g, 2.93 mmol) in THF (15 mL) is cooled to −78° C. n-Butyllithium (2.5 M in hexanes, 1.2 mL, 3.08 mmol) is added dropwise and the mixture is stirred for 1 h. (R)-(−)-glycidyl butyrate (0.44 mL, 3.08 mmol) is added dropwise and the mixture is stirred for 1 h. The cold bath is then removed and the mixture is stirred at RT for 48 h. The reaction is quenched with satd NH₄Cl and the aq layer is extracted with CH₂Cl₂. The org layer is washed with water, brine, dried over Na₂SO₄ and conc in vacuo. Purification by silica gel chromatography afforded 0.223 g of the title compound and 0.242 g of the butyric acid 3-(3-methyl-benzo[d]isoxazol-6-yl)-2-oxo-oxazolidin-5(R)-ylmethyl ester. The butyrate ester (0.242 g) is stirred with K₂CO₃ (0.500 g), CH₃OH (15 mL) and H₂O (5 mL) for 15 minutes and then conc in vacuo. The residue is diluted with CH₂Cl₂, washed with water (2×), brine, dried over Na₂SO₄ and conc in vacuo to give an additional 151 mg of the title compound for a total of 0.374 g (51%). ¹H NMR (400 MHz, DMSO-d₆): δ 7.84 (m, 2H), 7.72 (dd, J=8.8, 1.6 Hz, 1H), 5.24 (t, J=5.4 Hz, 1H), 4.76 (m, 1H), 4.19 (t, J=9.2 Hz, 1H), 3.94 (dd, J=8.8, 6.4 Hz, 1H), 3.71 (m, 1H), 3.59 (m, 1H), 2.53 (s, 3H). MS-APCI (m/z+): 249 (M+1). Anal. Calcd for C₁₂H₁₂N₂O₄: C, 58.06; H, 4.87; N, 11.28. Found: C, 58.37; H, 4.83; N, 11.09.

### Example 4 Preparation of (R)-3-(3-methyl-benzo[d]isoxazol-6-yl)-5-[1,2,3]triazol-1-ylmethyl-oxazolidin-2-one

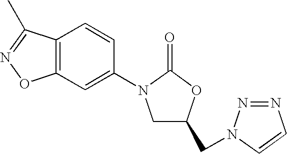

### Step 1: Preparation of (R)-Methanesulfonic acid 3-(3-methyl-benzo[d]isoxazol-6-yl)-2-oxo-oxazolidin-5-ylmethyl ester

Methanesulfonyl chloride (0.16 mL, 2.00 mmol) and triethylamine (0.4 mL, 2.85 mmol) are added to a 0° C. solution of 5(R)-hydroxymethyl-3-(3-methyl-benzo[d]isoxazol-6-yl)-oxazolidin-2-one (17) (0.354 g, 1.43 mmol) in CH₂Cl₂ (7 mL). After stirring at 0° C. for 30 min, the reaction is quenched with water and the layers are separated. The org layer is washed with water (2×), brine, dried over Na₂SO₄ and conc in vacuo. Purification by silica gel chromatography gave 0.369 g (79%) of the title compound. ¹H NMR (400 MHz, DMSO-d₆): δ 7.87 (d, J=9.0 Hz, 1H), 7.83 (d, J=1.6 Hz, 1H), 7.71 (dd, J=9.0, 1.6 Hz, 1H), 5.06 (m, 1H), 4.52 (m, 2H), 4.30 (t, J=9.6 Hz, 1H), 3.95 (dd, J=9.6, 6.4 Hz, 1H), 3.26 (s, 3H), 2.54 (s, 3H). MS-APCI (m/z+): 327 (M+1).

### Step 2: Preparation of (R)-5-azidomethyl-3-(3-methyl-benzo[d]isoxazol-6-yl)-oxazolidin-2-one

Sodium azide (0.28 g, 4.3 mmol) is added to methanesulfonic acid 3-(3-methyl-benzo[d]isoxazol-6-yl)-2-oxo-oxazolidin-5(R)-ylmethyl ester (0.369 g, 1.13 mmol) in DMF (10 mL) and the resultant mixture is heated to 75° C. overnight. After cooling to RT, EtOAc and H₂O are added and the phases are separated. The organic layer is washed with water (3×), brine, dried over Na₂SO₄ and conc in vacuo to give a quantitative yield (0.315 g) of the title compound. ¹H NMR (400 MHz, DMSO-d₆): δ 7.86 (d, J=8.8 Hz, 1H), 7.83 (d, J=2.0 Hz, 1H), 7.71 (dd, J=8.8, 2.0 Hz, 1H), 4.94 (m, 1H), 4.25 (t, J=9.2 Hz, 1H), 3.90 (dd, J=9.2, 6.4 Hz, 1H), 3.80 (dd, J=13.6, 3.2 Hz, 1H), 3.73 (dd, J=13.6, 5.4 Hz, 1H), 2.54 (s, 3H). MS-APCI (m/z+): 274 (M+1).

### Step 3: Preparation of (R)-3-(3-methyl-benzo[d]isoxazol-6-yl)-5-[1,2,3]triazol-1-ylmethyl-oxazolidin-2-on

3-(3-Methyl-benzo[d]isoxazol-6-yl)-5(R)-[1,2,3]triazol-1-ylmethyl-oxazolidin-2-one is prepared based on the procedure of Gravestock, M. B.; et al. PCT Int. Appl. WO 0181350, 2001: A mixture of 5(R)-azidomethyl-3-(3-methyl-benzo[d]isoxazol-6-yl)-oxazolidin-2-one (0.100 g, 0.366 mmol) and 2,5-norbornadiene (0.19 mL, 1.83 mmol) in dioxane (2 mL) is heated to reflux for 3 h. After cooling to RT, water and CH₂Cl₂ are added and the phases are separated. The org layer is washed with water (2×), brine, dried over Na₂SO₄ and conc in vacuo. Purification by silica gel chromatography gave 0.045 g (41%) of the title compound. ¹H NMR (400 MHz, DMSO-d₆): δ 8.18 (d, J=0.8 Hz, 1H), 7.84 (dd, J=8.0, 0.4 Hz, 1H), 7.76 (m, 2H), 7.61 (dd, J=8.8, 1.6 Hz, 1H), 5.19 (m, 1H), 4.87 (d, J=4.8 Hz, 2H), 4.35 (t, J=9.2 Hz, 1H), 4.01 (dd, J=9.2, 5.6 Hz, 1H), 2.53 (s, 3H). MS-APCI (m/z+): 300 (M+1), 256, 189.

### Example 5 Preparation of (R)-3-(3-methyl-benzo[d]isoxazol-6-yl)-5-(4-trimethylsilanylethynyl-[1,2,3]triazol-1-ylmethyl)-oxazolidin-2-one

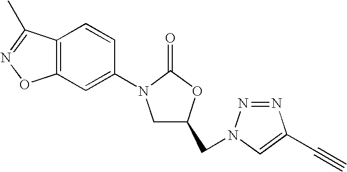

### Step 1: Preparation of (R)-methanesulfonic acid 3-(3-methyl-benzo[d]isoxazol-6-yl)-2-oxo-oxazolidin-5-ylmethyl ester

3-(3-Methyl-benzo[d]isoxazol-6-yl)-5(R)-(4-trimethylsilanylethynyl-[1,2,3]triazol-1-ylmethyl)-oxazolidin-2-one is prepared based on the procedure of Reck, F.; et al. J. Med. Chem. 2005, 48, 499-506: Methyllithium lithium bromide complex (1.5 M in ether, 5.1 mL, 7.7 mmol) is added to a solution of 1,4-bis(trimethylsilyl)butadiyne (1.00 g, 5.14 mmol) in ether (20 mL). After stirring overnight at RT, water is added and the layers are separated. The org layer is washed with sat NH₄Cl, brine, dried over Na₂SO₄ and conc in vacuo (130 torr) to give buta-1,3-diynyl(trimethyl)silane. A mixture of buta-1,3-diynyl(trimethyl)silane (0.112 g, 0.915 mmol), 5(R)-azidomethyl-3-(3-methyl-benzo[d]isoxazol-6-yl)-oxazolidin-2-one (0.100 g, 0.366 mmol), acetonitrile (2.5 mL), 2,6-lutidine (50 mL, 0.403 mmol), and copper (I) iodide (0.020 g, 0.105 mmol) is stirred at RT overnight. Water is added and the mixture is stirred for several minutes. The resultant solid is collected by filtration and washed with water and ether. Purification by silica gel chromatography afforded 0.080 g (55%) of the title compound. ¹H NMR (400 MHz CDCl₃): δ 7.86 (s, 1H), 7.55 (m, 2H), 7.44 (dd, J=8.8, 2.0 Hz, 1H), 5.07 (m, 1H), 4.76 (m, 2H), 4.24 (t, J=9.2 Hz, 1H), 3.97 (dd, J=9.2, 6.6 Hz, 1H), 2.53 (s, 3H), 0.22 (s, 9H). MS-APCI (m/z+): 396 (M+1).

### Step 2: Preparation of (R)-3-(3-methyl-benzo[d]isoxazol-6-yl)-5-(4-trimethylsilanylethynyl-[1,2,3]triazol-1-ylmethyl)-oxazolidin-2-one

Tetrabutylammonium fluoride (1.0 M in THF, 0.2 mL, 0.18 mmol) is added dropwise to a solution of 3-(3-methyl-benzo[d]isoxazol-6-yl)-5(R)-(4-trimethylsilanylethynyl-[1,2,3]triazol-1-ylmethyl)-oxazolidin-2-one (0.063 g, 0.159 mmol) in THF (3 mL). After stirring at RT for 2 h, the reaction is quenched with sat NaHCO₃ and the aq layer is extracted with CH₂Cl₂. The org layer is washed with brine, dried over Na₂SO₄, and conc in vacuo. Purification by prep HPLC gave 0.010 g (19%) of the title compound. ¹H NMR (400 MHz, CDCl₃): δ 7.90 (s, 1H), 7.58 (m, 2H), 7.47 (dd, J=8.8, 2.0 Hz, 1H), 5.09 (m, 1H), 4.77 (m, 2H), 4.27 (t, J=9.2 Hz, 1H), 4.01 (dd, J=9.2, 6.6 Hz, 1H), 3.24 (s, 1H), 2.55 (s, 3H). MS-APCI (m/z+): 324 (M+1).

### Example 6 Preparation of (R)-3-(3-methyl-benzo[d]isoxazol-6-yl)-2-oxo-oxazolidine-5-carboxylic acid amide

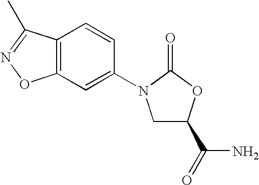

### Step 1: Preparation of 2(R)-hydroxy-3-(3-methyl-benzo[d]isoxazol-6-ylamino)-propionic acid methyl ester

Methyl (2R)-glycidate (0.1 mL, 1.21 mmol) is added to a solution of 6-amino-3-methyl-1,2-benzisoxazole (0.17 g, 1.15 mmol) in CH₃CN (2.5 mL). The mixture is heated to 50° C. and lithium trifluoromethanesulfonate (0.188 g, 1.20 mmol) is added in one portion. The mixture is then heated to 70° C. overnight. After cooling to RT, water and CH₂Cl₂ are added and the layers are separated. The org layer is washed with water (2×) and the aq layer is back-extracted with CH₂Cl₂. The combined org layer is washed with brine, dried over Na₂SO₄ and conc in vacuo. Purification by silica gel chromatography gave 0.188 g (65%) of the title compound. ¹H NMR (400 MHz, CDCl₃): δ 7.27 (d, J=8.8 Hz, 1H), 6.61 (d, J=2.0 Hz, 1H), 6.57 (dd, J=8.8, 2.0 Hz, 1H), 4.44 (dd, J=6.0, 3.6 Hz, 1H), 3.76 (s, 3H), 3.57 (dd, J=13.2, 3.6 Hz, 1H), 3.46 (dd, J=13.2, 6.0 Hz, 1H), 2.42 (s, 3H). MS-APCI (m/z+): 251 (M+1).

### Step 2: Preparation of (R)-3-(3-methyl-benzo[d]isoxazol-6-yl)-2-oxo-oxazolidine-5-carboxylic acid methyl ester

A solution of 2(R)-hydroxy-3-(3-methyl-benzo[d]isoxazol-6-ylamino)-propionic acid methyl ester (0.188 g, 0.751 mmol), 1,1′-carbonyldiimidazole (0.183 g, 1.13 mmol) and CH₃CN (5 mL) is stirred at RT overnight. The mixture is then heated to 55° C. and stirred overnight. After cooling to RT, water and CH₂Cl₂ are added and the layers are separated. The aq layer is extracted with CH₂Cl₂ and the combined org layer is washed with water, brine, dried over Na₂SO₄ and conc in vacuo. A mixture of CH₂Cl₂ and hexanes is added to the residue and the mixture is cooled to 0° C. The resultant precipitate is collected by filtration to give 0.096 g (46%) of the title compound, which is used without further purification. ¹H NMR (400 MHz, CDCl₃): δ 7.62 (m, 3H), 5.10 (m, 1H), 4.36 (m, 1H), 4.22 (m, 1H), 3.87 (s, 3H), 2.55 (s, 3H). MS-APCI (m/z+): 277 (M+1).

### Step 3: Preparation of (R)-3-(3-methyl-benzo[d]isoxazol-6-yl)-2-oxo-oxazolidine-5-carboxylic acid amide

A suspension of 3-(3-methyl-benzo[d]isoxazol-6-yl)-2-oxo-oxazolidine-5(R)-carboxylic acid methyl ester (0.096 g, 0.346 mmol), 2 M NH₃ in CH₃OH (2.0 mL), and CH₃CN (2 mL) is sired at RT overnight. The mixture is conc in vacuo, diluted with CH₃OH and cooled to 0° C. for 30 min. The resultant solid is collected by filtration to give 0.056 g (62%) of the title compound. ¹H NMR (400 MHz, DMSO-d₆): δ 7.89 (br s, 1H), 7.86 (m, 2H), 7.74 (m, 1H), 7.64 (br s, 1H), 5.08 (dd, J=9.6, 6.1 Hz, 1H), 4.38 (dd, J=9.6, 9.2 Hz, 1H), 4.12 (dd, J=9.2, 6.1 Hz, 1H), 2.54 (s, 3H). Anal. Calcd for C₁₂H₁₁N₃O₄: C, 55.17; H, 4.24; N, 16.08. Found: C, 54.97; H, 4.10; N, 15.85.

### Example 7 Preparation of (R)-3-(3-methyl-benzo[d]isoxazol-6-yl)-2-oxo-oxazolidine-5-carboxylic acid methylamide

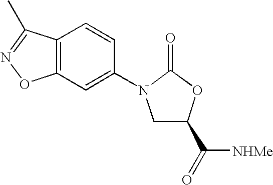

A slurry of 3-(3-methyl-benzo[d]isoxazol-6-yl)-2-oxo-oxazolidine-5(R)-carboxylic acid methyl ester (0.130 g, 0.471 mmol) and 2 M methylamine in CH₃OH (2.4 mL) is stirred at RT for 4 h. The mixture is conc in vacuo, diluted with CH₃OH and cooled to 0° C. for 1 h. The resultant solid is collected by filtration and washed with CH₃OH to give 0.114 g (88%) of the title compound. ¹H NMR (400 MHz, DMSO-d₆): δ 8.42 (br d, J=4.2 Hz, 1H), 7.86 (m, 2H), 7.73 (m, 1H), 5.11 (dd, J=9.6, 5.8 Hz, 1H), 4.39 (dd, J=9.6, 9.2 Hz, 1H), 4.13 (dd, J=9.2, 5.8 Hz, 1H), 2.67 (d, J=4.2 Hz, 3H), 2.54 (s, 3H). MS-APCI (m/z+): 276 (M+1). Anal. Calcd for C₁₃H₁₃N₃O₄: C, 56.73; H, 4.76; N, 15.27. Found: C, 56.58; H, 4.55; N, 15.12.

### Example 8 Preparation of (R)-3-(3-methyl-benzo[d]isoxazol-6-yl)-2-oxo-oxazolidine-5-carboxylic acid methoxy-amide

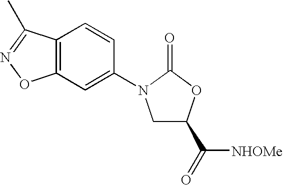

A mixture of 3-(3-methyl-benzo[d]isoxazol-6-yl)-2-oxo-oxazolidine-5(R)-carboxylic acid methyl ester (0.130 g, 0.471 mmol), methoxylamine hydrochloride (0.197 g, 2.35 mmol) and pyridine (4 mL) is heated to 70° C. overnight. An additional 0.197 g of methoxylamine hydrochloride is added and the mixture is again stirred at 70° C. overnight. After cooling to RT, H₂O and CH₂Cl₂ are added and the phases are separated. The org layer is washed with 10% aq citric acid, brine, dried over Na₂SO₄ and conc in vacuo to give 0.097 g (71%) of the title compound. ¹H NMR (400 MHz, DMSO-d₆): δ 11.83 (br s, 1H), 7.86 (m, 2H), 7.74 (m, 1H), 5.06 (dd, J=9.2, 5.6 Hz, 1H), 4.38 (dd, J=9.6, 9.2 Hz, 1H), 4.18 (dd, J=9.6, 5.6 Hz, 1H), 3.65 (s, 3H), 2.54 (s, 3H). Anal. Calcd for C₁₃H₁₃N₃O₅: C, 53.61; H, 4.50; N, 14.43. Found: C, 53.59; H, 436; N, 14.33.

### Example 9 Preparation of (R)-3-(3-methyl-benzo[d]isoxazol-6-yl)-2-oxo-oxazolidine-5-carboxylic acid ethoxy-amide

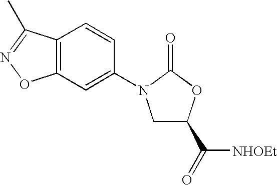

A mixture of 3-(3-methyl-benzo[d]isoxazol-6-yl)-2-oxo-oxazolidine-5(R)-carboxylic acid methyl ester (0.130 g, 0.471 mmol), O-ethylhydroxylamine hydrochloride (0.230 g, 2.35 mmol) and pyridine (4 mL) is heated to 70° C. overnight. An additional 0.230 g of O-ethylhydroxylamine hydrochloride is added and the mixture is again stirred at 70° C. overnight. After cooling to RT, H₂O and CH₂Cl₂ are added and the phases are separated. The org layer is washed with 10% aq citric acid (3×), water, brine, dried over Na₂SO₄ and conc in vacuo to give 0.057 g (39%) of the title compound. ¹H NMR (400 MHz, DMSO-d₆): δ 11.70 (br s, 1H), 7.86 (m, 2H), 7.74 (dd, J=8.8, 2.0 Hz, 1H), 5.05 (dd, J=9.6, 5.6 Hz, 1H), 4.38 (t, J=9.2 Hz, 1H), 4.17 (dd, J=9.6, 5.6 Hz, 1H), 3.87 (q, J=7.2 Hz, 2H), 2.54 (s, 3H), 1.17 (t, J=7.2 Hz, 3H). Anal. Calcd for C₁₄H₁₅N₃O₅: C, 55.08; H, 4.95; N, 13.76. Found: C, 54.85; H, 4.79; N, 13.58.

We claim:

1. A compound of formula I 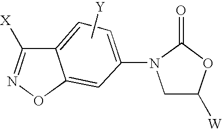
or a pharmaceutically acceptable salt thereof wherein:
W is
(a) CH₂NHC(═Z)R¹,
(b) C(═Z)NHR², or
(c) CH₂het;
X is H, C₁₋₆alkyl, or C₂alkenyl;
Y is H, or F;
Z is O, or S;
R¹ is
(a) C₁₋₆alkyl,
(b) NHC₁₋₆alkyl,
(c) C₃₋₇cycloalkyl,
(d) C₂₋₆alkenyl, or
(e) OC₁₋₄alkyl;
R² is
(a) H,
(d) C₁₋₄alkyl, or
(e) —OC₁₋₄alkyl; and
het is a five-(5) or six-(6) membered heterocyclic ring having 1-4 heteroatoms selected from the group consisting of oxygen, sulfur, and nitrogen within the ring, wherein each carbon atom in het is optionally substituted with C₁₋₄alkyl, C₂₋₄alkenyl, C₂₋₄alkynyl, halo, OR³, CN, NO₂, NHR³R³, oxo, CF₃, OCF₃, C(═O)C₁₋₄alkyl, OC(═O)C₁₋₄alkyl, or C(═O)OR³; wherein R³ is H, or C₁₋₄alkyl.

2. A compound of claim 1 wherein W is CH₂NHC(═O)C₁₋₂alkyl, or CH₂NHC(═O)OC₁₋₂alkyl,

3. A compound of claim 1 wherein W is C(═O)NHC₁₋₂alkyl, or C(═O)NHOC₁₋₂alkyl.

4. A compound of claim 1 wherein W is 1,2,3-triazole-1-yl methyl.

5. A compound of claim 1 which is
(1) (S)—N-[3-(3-methyl-benzo[d]isoxazol-6-yl)-2-oxo-oxazolidin-5-ylmethyl]-acetamide,
(2) (S)—N-[3-(3-methyl-benzo[d]isoxazol-6-yl)-2-oxo-oxazolidin-5-ylmethyl]-propionamide,
(3) (S)-[3-(3-methyl-benzo[d]isoxazol-6-yl)-2-oxo-oxazolidin-5-ylmethyl]-carbamic acid methyl ester,
(4) (R)-3-(3-methyl-benzo[d]isoxazol-6-yl)-5-[1,2,3]triazol-1-ylmethyl-oxazolidin-2-one,
(5) (R)-3-(3-methyl-benzo[d]isoxazol-6-yl)-5-(4-trimethylsilanylethynyl-[1,2,3]triazol-1-ylmethyl)-oxazolidin-2-one,
(6) (R)-3-(3-methyl-benzo[d]isoxazol-6-yl)-2-oxo-oxazolidine-5-carboxylic acid amide,
(7) (R)-3-(3-methyl-benzo[d]isoxazol-6-yl)-2-oxo-oxazolidine-5-carboxylic acid methylamide,
(8) (R)-3-(3-methyl-benzo[d]isoxazol-6-yl)-2-oxo-oxazolidine-5-carboxylic acid methoxy-amide, or
(9) R)-3-(3-methyl-benzo[d]isoxazol-6-yl)-2-oxo-oxazolidine-5-carboxylic acid ethoxy-amide.

6. A pharmaceutical composition comprising a compound of claim 1 or a pharmaceutically acceptable salt thereof, and a pharmaceutically acceptable carrier.

7. A method for treating bacteria infections comprising administering to a mammal being treated a pharmaceutically effective amount of the compound of claim 1.

8. The method of claim 7 wherein the compound of claim 1 is administered orally, parenterally, topically, rectally, or intranasally.

9. The method of claim 7 wherein said compound is administered in an amount of from about 0.1 to about 100 mg/kg of body weight/day.

10. The method of claim 7 wherein said compound is administered in an amount of from about 1 to about 50 mg/kg of body weight/day.

11. The bacteria infection of claim 7 which is ear infections, eye infections, respiratory tract infections, skin and skin structure infections, bacterial endocarditis, osteomyelitis, endocarditis or diabetic foot.

12. The bacteria infection of claim 7 which is caused by gram-positive bacteria, gram negative bacteria, anaerobic organisms, and acid-fast organisms.

13. The bacteria infection of claim 7 which is caused by bacteria comprising staphylococci, streptococci, Enterococci, Haemophilus, Moraxella, bacteroides, clostridia, Mycobacteria, or Chlamydia.

14. The bacteria of claim 13 wherein staphylococci is S. aureus and S. epidermidis; wherein streptococci is S. pneumoniae of S. pyogenes; wherein Enterococci is E. faecalis; wherein Haemophilus is H. influenzae; wherein Moraxella is M. catarrhalis; and wherein Mycobacteria is M. tuberculosis; or Mycobacterium avium.

15. The bacteria infections of claim 7 which is caused by multi-dug resistant S. aureus.
